(19) United States
(12) Patent Application Publication
(10) Pub. No.: US 2018/0177520 A1
(43) Pub. Date: Jun. 28, 2018

(54) Minimally Invasive Focused Ultrasound (MIFUS) for Brain Surgery

(71) Applicant: THE JOHNS HOPKINS UNIVERSITY, Baltimore, MD (US)

(72) Inventor: Peter Miller, Silver Spring, MD (US)

(21) Appl. No.: 15/900,999

(22) Filed: Feb. 21, 2018

Related U.S. Application Data
(63) Continuation of application No. 14/207,181, filed on Mar. 12, 2014, now abandoned.
(60) Provisional application No. 61/781,032, filed on Mar. 14, 2013.

(51) Int. Cl. A61B 17/32; A61B 1/07

(52) U.S. Cl. CPC A61B 17/320068; A61B 1/07

(57) ABSTRACT

HIFU (High-Intensity Focused Ultrasound) sometimes FUS or MIFUS is a highly precise medical procedure using high-intensity focused ultrasound to heat and destroy pathogenic tissue rapidly in the brain for neurosurgical purposes, by causing coagulation necrosis. Here we have described a system that uses specially designed micro-transducer heads that are placed inside the later ventricles for the purposes of MIFUS lesioning of brain targets under real time MRI, endoscopic and Doppler guidance. A minimally invasive approach using Kocher's point allows small incisions with little bleeding, recovery time, infection risk and surgical time. This approach allows insertion of the micro-ultrasound transducers into the lateral ventricles of the brain avoiding skull bone attenuation of the ultrasonic waves and unnecessary heating of brain tissues in the process.

### CROSS-REFERENCES

1. Neurosurgery. 2009 February; 64(2):201-10; discussion 210-1. High-intensity focused ultrasound surgery of the brain: part 1—A historical perspective with modern applications. Jagannathan J, Sanghvi N T, Crum L A, Yen C P, Medel R, Dumont A S, Sheehan J P, Steiner L, Jolesz F, Kassell N F.
2. Neurosurgery. 2006 November; 59(5):949-55; discussion 955-6. Magnetic resonance imaging-guided, high-intensity focused ultrasound for brain tumor therapy. Ram Z, Cohen Z R, Harnof S, Tal S, Faibel M, Nass D, Maier S E, Hadani M, Mardor Y.
3. Feasibility of miniature high frequency piezoelectric ceramic hollow spheres for exposimetry and tissue ablation. Osama M et al. 2003
4. Bouras T, Sgouros S. Complications of endoscopic third ventriculostomy. J Neurosurg Pediatr. 2011 June; 7(6):643-9.
5. Werner J, Park E J, Lee H, Francischelli D, Smith N B. Feasibility of in vivo transesophageal cardiac ablation using a phased ultrasound array. Ultrasound Med Biol. 2010 May; 36(5):752-60. Epub 2010 Mar. 28.

### BACKGROUND AND RELATED ART

History of Focused UltraSound (FUS) in the Human Brain

The evolution of Magentic Resonance Guided Focused Ultrasound Surgery (MRgFUS) has occurred in parallel with modern neurological surgery. Early studies on focused ultrasound treatment of brain tumors in the 1940s and 1950s, demonstrated the ability to perform precise lesioning in the human brain, with a favorable risk-benefit profile. The need for a craniotomy, as well as the lack of sophisticated imaging technology, resulted in limited growth of high-intensity focused ultrasound for neurosurgery. More recent, technological advances have permitted the combination of high-intensity focused ultrasound along with magnetic resonance imaging guidance to provide an opportunity to effectively treat a variety of central nervous system disorders. [1] Despite anticipation, the newer technology suffers from several major disadvantages which significantly interfere with therapy and/or has not yet met minimum safety standards. Although work goes on to get approval for these devices, the door remains open for a comprehensive technological answer to this problem.

There is an ongoing need simple, safe, relatively inexpensive, minimally invasive system to deliver focused ultrasonic energy to targets inside the brain. Stated another way “Hands free method to destroy brain lesions without massive craniotomies entailing needless infection, blood loss, wound healing and even death”

### SUMMARY OF EMBODIMENTS

As noted above, there is an ongoing need simple, safe, relatively inexpensive, minimally invasive system to deliver focused ultrasonic energy to targets inside the brain. Stated another way “Hands free method to destroy brain lesions without massive craniotomies entailing needless infection, blood loss, wound healing and even death”

Not wishing to be bound by theory, it is noted that the skull bone attenuates ultrasonic energy and becomes hot which is a high Risk to patients. Presence of such heat increases the time of surgery due to intermittent periods for cooling.

Single trajectory approaches may be problematic due to the fact that multiple angles of approach are needed (multiple transducer heads) to destroy lesions completely. There is a need to be non or minimally invasive and to remove little to no bone. It is preferred to avoid any collateral damage to any brain or blood vessels. With ultrasonic transducers placed outside the brain, many areas of the brain are at risk for damage.

The present disclosure incorporates by reference U.S. 61/781,032 filed on or around Mar. 14, 2013 and entitled “MINIMALLY INVASIVE FOCUSED ULTRASOUND (FUS) FOR NEURAL ABLATIVE THERAPIES.” In some embodiments, any feature or combination of features described in the present document may be combined with any feature of combination of features described in application U.S. 61/781,032.

HIFU (High-Intensity Focused Ultrasound) sometimes FUS or MIFUS is a highly precise medical procedure using high-intensity focused ultrasound to heat and destroy pathogenic tissue rapidly in the brain for neurosurgical purposes, by causing coagulation necrosis. Here we have described a system that uses specially designed micro-transducer heads that are placed inside the later ventricles for the purposes of MIFUS lesioning of brain targets under real time MRI, endoscopic and Doppler guidance. A minimally invasive approach using Kocher's point allows small incisions with little bleeding, recovery time, infection risk and surgical time. This approach allows insertion of the micro-ultrasound transducers into the lateral ventricles of the brain avoiding skull bone attenuation of the ultrasonic waves and unnecessary heating of brain tissues in the process. A catheter cooled endoscopic approach with a fiberoptic tube inside an expandable mesh sheath that allows UV light transmittance for a sterile working field is used to position and control these transducer heads in the ventricles. Sophisticated computer software allows real time surgical planning and guidance and MRI and Dopper provide real time temperature and blood flow of brain and target tissues. The system is easy to place and easy to use with minimal room for operator error. A transducer may be even left inside the ventricle a “pill” form for use at a later time, remotely controlled. This system can be used for brain tumors, clot lysis in stroke, epilepsy, deep brain stimulation, a drug delivery systems among many other uses.

It is now disclosed a system and surgical technique for minimally invasive neurological surgery that uses focused ultrasound lesioning of brain tumors from within the lateral ventricles of the brain using real time MRI guidance. The system is composed of: (i) micro-ultrasound transducers that project the focused ultrasound energy and are designed to be used from inside the brains lateral, third or fourth ventricle systems. These transducers also have the following capabilities:
Very small 1-5 mm or less
Movable/steerable in 3 dimensions
Can be broken into small parts that are inserted piecemeal into the ventricle system
Can be automatically assembled inside lateral ventricle in real time surgery using magnetic or mechanical locking mechanisms
Closed loop cooling circuitry
Can be covered with a special polycarbonate material that has a high heat capacity and resists heating is MRI compatible and waterproof.
Can provide sufficient energy intensity and focal length to reach and safely destroy targets within the brain as shown in FIG. 10.
Can be assembled inside ventricle, see FIG. 5; (waterproof covering)
Powerful enough to provide up to 50 W/cm2 intensity
MRI compatible materials.
Can be inserted as a “pill” single small unit that is remotely controlled and powered for using focused ultrasound (FUS) in brain.

(ii) Use one or both lateral ventricles for targeting the brain or brain lesions with high intensity focused ultrasound technology. This system and method depends on the use of the one or both simultaneously, lateral ventricles as a staging point for origination of the focused ultrasonic energy from the transducer heads placed inside them. What is claimed here is the lateral ventricles use for containing transducer heads that emit high intensity or other focused ultrasonic energy for heating brain targets. The lateral ventricles are used as a staging point for the positioning, assembly and use of the heads for heating brain targets using focused ultrasound. The empty ventricles provide room to work, to fire the ultrasonic energy, a protective covering of the brain during surgery and a method to avoid the attenuation of ultrasonic energy by the skull bone which completely surrounds the brain.

(iii) A system that makes use of currently available video (ventricle endoscope), MRI and ultrasound with Doppler for real time positioning, thermal dose delivery control and visual data feedback of transducer action, see FIG. 10. The MRI will provide real time surgical thermal information of the brain, target and transducers. The endoscope will be inserted into the ventricle to guide transducer positioning and construction in real time surgery. The Doppler and ultrasound probe will be inserted into the ventricle and provide real time blood flow imaging of the target vessels.

(iv) Targeting computer, with specially designed software that calculates the amount of thermal energy needed to destroy the target based in target density and size measurements from preoperative imaging. The computer then does the following:
A. Provides the surgeon with a choice of use of different size micro-tranducer heads alone or in tandem to provide this energy, based on the amount of energy needed per head, the time and the heating of the heads in a way which allows little to no heat dispersion to brain tissue surrounding the ventricle where the transducers work.
B. Can allow use of one or multiple transducer heads in the later ventricle or one or multiple transducer heads in tandem from both lateral ventricles simultaneously.
C. Can provide a 3D map of where to position these heads in real time during surgery within the lateral ventricle or both lateral ventricles to achieve the desired lesioning of the target during the timeline of surgery.
D. Can allow the surgeon to design how he wants to lesion the target either piecemeal or all at once or from inside to outside etc.
E. Provides a guidance program during the entire surgical lesioning process displaying step by step instructions to the surgeon and giving real time data feedback of the target (temperature, size , blood flow and so forth).
F. Can allow for on the fly changes to the surgical program in coordination with the surgeons wishes.
G. Records the entire surgical process for further review and potential research.

In general the software with computer will have the following capabilities:
Construction Software (used for transducer assembly inside skull);
Assembly plan program calculates most efficient plan for a given surgery;
Component program for calculation of transducer number and other needed surgical elements;
Real time ventricular video images guiding plan operation in real time;
Targeting Software (guides energy to precise areas of target at precise times) used for:
Navigation Software—Distance from transducer to target;
Positioning of Heads;
Ablation program—Depends on blood flow to target, surgeons desires etc. Determines which areas will be ablated first and how;
Determining needed frequency for ablation program steps;
Ablation Monitoring Software;
Displays color coded temperature map of targets;
Displays a 3D real time image of target as it is destroyed;
Displays ultrasound and Doppler blood flow to target;
Displays color coded map of transducers.

(v) Minimally invasive surgical method which involves a small incision in the scalp, cautization of bleeders, a 12 mm or larger standard size burr hole being drilled in the skull bone, incision to open the dura, and insertion of a mechanically expandable (up to 12 mm or more) flexible sheath into the lateral ventricle. Our system will utilize the Kocher's point which is 2-3 cm lateral to the midline of the skull and 1-2 cm anterior to the coronal suture, see FIG. 2 because of the following reasons:
Position on skull which gives shortest direct line distance to the lateral ventricles
Positions device in larger area (anterior horn) of the ventricle but also avoids all major blood vessels and eloquent neural strips
Allows insertion of sheath into lateral ventricle with no damage to brain or vessels.

In some embodiments other surgical positions or configurations will work for this system. Any position on the skull may be used and any type of hole may be drilled 1 mm to larger burr hole 12 mm size. In the most basic form however, a small hole drilled at Kocher's point according to the method used for a beside ventriculostomy procedure, and insertion of the expandable sheath into the ventricle, and afterwards insertion of all of the transducer heads into the lateral ventricles.

(vi) Restraining bolt: A specially designed plastic frameless system that is screwed to the skull at Kocher's point for the purposes of securing the expandable sheath as well as the other instruments inserted into the later ventricles and also providing trajectory guidance during the insertion of the sheath into the lateral ventricle (see FIG. 8).

(vii) Expandable entry port sheath for access to the brain via Kocher's Point. This sheath will be a mesh tube type design that is mechanically expandable (up to 12 mm or more), size locked into place, flexible and can be inserted into the lateral ventricles. In addition it will have:
A 3 mm mesh tube which can be expanded up to 1.2-1.5 cm diameter and is used as a brain entry port once the burr holes and ventricle tap have been finished
A fiber optic inner liner which can be inserted into the tube which will have UV light transmitted intermittently or continuously during surgery brain down to the inside of the ventricle to sterilize the area and prevent operative infection.

The term ‘minimally invasive neurosurgery’ as opposed to ‘non-invasive neurosurgery’ means that a small incision is made, and a small hole is drilled in the skull (12 mm) in order to get access to the lateral ventricle for positioning of MIFUS instrumentation. There is very little blood loss, infection and recovery time with minimally invasive neurosurgery. This is as opposed to non-invasive which involves no incision and is done completely outside the skull. This is as opposed to invasive which uses large incisions and removal of a large amount of bone to gain access to the brain such as is common in craniotomies.

The term frameless means, that a ‘stereotactic frame’ system as is typically employed in neurosurgery does not need to be used here for insertion of instruments into the lateral ventricle. A frameless system is simply attached to the skull mechanically and serves as a secure point and trajectory guidance system for insertion of MIFUS instruments.

In some embodiments the transducer heads may be inserted into both lateral ventricles simultaneously and may work in tandem to lesion brain targets.

In some embodiments the no restraining bolt will be used and no burr holes will be drilled. A simple small incision will be placed in the scalp and a hand drill used to place a small hole in the skull bone and dura through which the expandable sheath and MIFUS instruments can be inserted and the lesioning process can continue from that point as has been described.

In some embodiments many different types and or sizes of transducer heads may be inserted into the lateral brain ventricles and they do not necessarily need to be inserted and constructed piecemeal inside, but rather can be inserted whole and then used according to MIFUS protocol.

In some embodiments a remotely controlled transducer head “pill” may be inserted into the patients lateral ventricle for long term recurrent therapy stretching over days to months. This pill will receive power from small batteries or RF induced or other externally generated energy. The pill can be powered remotely and can direct focused ultrasound under MRI guidance to targets in the brain.

In some embodiments the endoscope or Doppler may not be used but rather only the transducer heads, expandable sheath and MRI and guidance computer.

In some embodiments the restraining bolt may be attached to the skull and then have an opening that rises 30 or more cm above that point for the purpose of securing MIFUS instrumentation in certain situations.

In some embodiments the MRI may be a smaller low resolution MRI such as a 1-1.5 tesla, that allows easy maneuvering inside the operating room and yet gives sufficient real time MRI visual data for the MIFUS brain lesioning process.

In some embodiments the computer and targeting system may be connected wirelessly to the MIFUS instruments attached and inserted into the patients later ventricles.

Some embodiments relate to micro-ultrasound transducer heads that project the focused ultrasound energy and are designed to be used from inside the brains lateral, third or fourth ventricle systems.

Some embodiments relate to remote assembly ability of transducer heads inside of the ventricle for MIFUS neurosurgery.

In some embodiemnts, the micro transducers listed have the one or more (i.e. any number of or any combination of, for example, any one of, or a plurality of, or a majority of, or all of) the following capabilities:
Very small 1-5 mm or less
Movable/steerable in 3 dimensions
Can be broken into small parts that are inserted piecemeal into the ventricle system
Can be broken apart inserted piecemeal into the lateral ventricle and then automatically assembled inside lateral ventricle in real time surgery using magnetic or mechanical locking mechanisms
Closed loop cooling circuitry using coolant water jacket approach
Can be covered with a special polycarbonate material that has a high heat capacity and resists heating is MRI compatible and waterproof.
Can provide sufficient energy intensity and focal length to reach and safely destroy targets within the brain as shown in FIG. 10.
Can be made of material is waterproof for use inside ventricle, see FIG. 5;
Powerful enough to provide up to 50 W/cm2 intensity
MRI compatible materials.
Can be inserted as a discrete “pill” single small unit that is remotely controlled and powered for using focused ultrasound (FUS) in brain.

Some embodiments relate to one or both lateral ventricles for targeting the brain or brain lesions with high intensity focused ultrasound technology.

Some embodiments relate to the use of the lateral ventricles as a staging point for origination “firing” of the focused ultrasonic energy from the transducer heads placed inside them.

Some embodiments relate to the use of the lateral ventricles of the brain for containing transducer heads that emit high intensity or other focused ultrasonic energy for heating brain targets. The lateral ventricles are used as a staging point for the positioning, assembly and use of the heads for heating brain targets using focused ultrasound. The empty ventricles provide room to work, to fire the ultrasonic energy, a protective covering of the brain during surgery and a method to avoid the attenuation of ultrasonic energy by the skull bone which completely surrounds the brain.

Some embodiments relate to use of video (e.g. ventricle endoscope), MRI and/or ultrasound with Doppler for real time positioning, thermal dose delivery control and visual data feedback of micro transducer action inside the lateral ventricles as, see FIG. 10.

Some embodiments relate to the use of MRI to provide real time surgical thermal information of the brain, target and transducers during their use inside the lateral ventricles. The endo scope will be inserted into the ventricle to guide transducer positioning and construction in real time surgery. The Doppler and ultrasound probe will be inserted into the ventricle and provide real time blood flow imaging of the target vessels.

Some embodiments relate to an electronic device (e.g. a targeting computer), for use in controlling the focused ultrasound dose to brain targets from micro transducers placed within the lateral ventricles of the brain. This software and computer will have the ability to calculate the amount of thermal energy needed to destroy the target based in target density and size measurements from preoperative imaging. The computer then does one or more (e.g. all of) the following:

A. Provides the surgeon with a choice of use of different size micro-tranducer heads alone or in tandem to provide this energy, based on the amount of energy needed per head, the time and the heating of the heads in a way which allows little to no heat dispersion to brain tissue surrounding the ventricle where the transducers work.

B. Can allow use of one or multiple transducer heads in the later ventricle or one or multiple transducer heads in tandem from both lateral ventricles simultaneously.

C. Can provide a 3D map of where to position these heads in real time during surgery within the lateral ventricle or both lateral ventricles to achieve the desired lesioning of the target during the timeline of surgery.

D. Can allow the surgeon to design how he wants to lesion the target either piecemeal or all at once or from inside to outside etc.

E. Provides a guidance program during the entire surgical lesioning process displaying step by step instructions to the surgeon and giving real time data feedback of the target (temperature, size, blood flow and so forth).

F. Can allow for on the fly changes to the surgical program in coordination with the surgeons wishes.

G. Records the entire surgical process for further review and potential research.

In some embodiments, software for control of the MIFUS process which has one or more of (e.g. majority of, e.g. al of) the following capabilities:

Construction Software (used for transducer assembly inside skull);

Assembly plan program calculates most efficient plan for a given surgery;

Component program for calculation of transducer number and other needed surgical elements;

Real time ventricular video images guiding plan operation in real time;

Targeting Software (guides energy to precise areas of target at precise times) used for:

Navigation Software—Distance from transducer to target;

Positioning of Heads;

Ablation program—Depends on blood flow to target, surgeons desires etc. Determines which areas will be ablated first and how;

Determining needed frequency for ablation program steps;

Ablation Monitoring Software;

Displays color coded temperature map of targets;

Displays a 3D real time image of target as it is destroyed;

Displays ultrasound and Doppler blood flow to target;

Displays color coded map of transducers.

It is now disclosed a minimally invasive surgical method for use with MIFUS which involves in the most basic form a small hole drilled at Kocher's point according to the method used for a beside ventriculostomy procedure, and insertion of the expandable sheath into the ventricle, and afterwards insertion of all of the transducer heads into the lateral ventricles. It may also involve a small incision in the scalp, cautization of bleeders, a 12 mm or larger standard size burr hole being drilled in the skull bone, incision to open the dura, and insertion of a mechanically expandable (up to 12 mm or more) flexible sheath into the lateral ventricle. Our system will utilize the Kocher's point which is 2-3 cm lateral to the midline of the skull and 1-2 cm anterior to the coronal suture, see FIG. 2 because of the following one or two or all of the following reasons:
Position on skull which gives shortest direct line distance to the lateral ventricles
Positions device in larger area (anterior horn) of the ventricle but also avoids all major blood vessels and eloquent neural strips
Allows insertion of sheath into lateral ventricle with no damage to brain or vessels.

It is now disclosed a restraining bolt which as defined here is a specially designed plastic frameless system that is screwed to the skull at Kocher's point for the purposes of securing the expandable sheath as well as the other instruments inserted into the later ventricles and also providing trajectory guidance during the insertion of the sheath into the lateral ventricle (see FIG. 8).

It is now disclosed the use of an neurosurgical ventricle endo scope for MIFUS neurosurgery with the system described above.

It is now disclosed the use of real time Doppler ultrasound to monitor blood flow to and from target for MIFUS neurosurgery purposes from within the lateral brain ventricles.

It is now disclosed an entry port sheath for access to the brain via Kocher's Point. This sheath will be a mesh tube type design that is mechanically expandable (up to 12 mm or more), size locked into place, flexible and can be inserted into the lateral ventricles. In addition it will have any one, or both of the following:
A 3 mm mesh tube which can be expanded up to 1.2-1.5 cm diameter and is used as a brain entry port once the burr holes and ventricle tap have been finished
A fiber optic inner liner which can be inserted into the tube which will have UV light transmitted intermittently or continuously during surgery brain down to the inside of the ventricle to sterilize the area and prevent operative infection.

### BRIEF DESCRIPTION OF THE DRAWINGS

FIG. 1 Image of difficult to avoid brain vasculature. This figure illustrates the potential difficulty in targeting a point in the brain.

FIG. 2 Kocher's Point Illustration. This figure shows the approximate position of the point on a patient's skull where the expandable sheath will be inserted.

FIG. 3 The MIFUS Overall Configuration. This is a system diagram of all of the major pieces of the MIFUS system and their basic connectivity.

FIG. 4 Relevant Areas of the Brain. The ventricular position in the brain that will be used is illustrated here.

FIG. 5 Micro Transducer (Zoom View) Construction. This figure shows a typical assembly of micro transducers at the far end of the expandable sheath after the sheath has been inserted into the brain of the patient.

FIG. 6 MRI/Patient Positioning Relationship. This shows how the patient will be placed on the MRI table in a stable position; and how adjustments can be made to the equipment if necessary.

FIG. 7 MRI Possible Configurations. Shown here is a possible position that the patient may have on the MRI table other than flat on the back.

FIG. 8 Kocher's Point Restraining Bolt. This illustrates the type of stabilization that will be used to hold the expandable sheath and associated wiring that goes through the sheath.

FIG. 9 Ventricular Equipment View. This figure gives a symbolic view (not to scale) of the components of MIFUS utilized inside the brain.

FIG. 10 Lesioning of Brain Tumor View. This figure is a variation of FIG. 9 which shows how the ultrasound waves will be focused on a brain tumor; and it shows how the Doppler ultrasound will be located to transmit positioning information to the computer.

### DETAILED DECSRIPTION OF EMBODIMENTS

Embodiments of the present invention relate to systems, methods and kits for heating targets in the brain using high intensity focused ultrasound fired from transducer heads placed inside the brains lateral ventricles using a minimally invasive approach all while under the real time imaging control of MRI, Doppler and visual systems.

The invention is herein described, by way of example only, with reference to the accompanying drawings. With specific reference now to the drawings in detail, it is stressed that the particulars shown are by way of example and for purposes of illustrative discussion of the preferred embodiments of the exemplary system only and are presented in the cause of providing what is believed to be a useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice and how to make and use the embodiments.

For brevity, some explicit combinations of various features are not explicitly illustrated in the figures and/or described. It is now disclosed that any combination of the method or device features disclosed herein can be combined in any manner—including any combination of features—any combination of features can be included in any embodiment and/or omitted from any embodiments.

In relation to the figures the following numbers indicate:

1—Kocher's surgical entry point

2—Expandable sheath

3—Brain parenchyma

4—Lateral ventricle

5—Expandable sheath with ultrasound probe

6—Ultrasound transducer head

7—Example of brain tumor target

8—Control computer and software

9—Relevant brain anatomy with ventricular system displayed inside

10—Example of disassembled transducer head parts that fit together inside

11—Example of assembled transducer which is movable in 3D inside the ventricle

12—MRI with device during MIFUS neurosurgery; one possible configuration

13—Cross section of MRI with patient during MIFUS neurosurgery

14—Skull Bone

15—Brain parenchyma exposed through a burr hole created in skull

16—Restraining bold frameless system

17—Top tube of restraining bold can be longer than implied by this simple drawing as needed for MIFUS instruments stability

18—One possible configuration of MIFUS instruments inserted for neurosurgery

19—One possible configuration of MIFUS instruments positioned in brain for surgery while connected to MRI and computer

This device is called Minimally Invasive Focused Ultrasound (MIFUS) for brain and it is a system of components that will take advantage of proven capabilities for such things as tumor ablation; but at the same time will utilize the brains lateral ventricle as an anatomical advantage and advanced ultrasound transducer micro technology. MIFUS neurosurgery in this context is defined as follows:
Brain surgery done with only a small incision and through a cannula often employing an endoscope
Little to no risk of bleeding and no damage to eloquent brain tissue;
Sterile hands free environment
Very brief surgical times as compared to craniotomies.

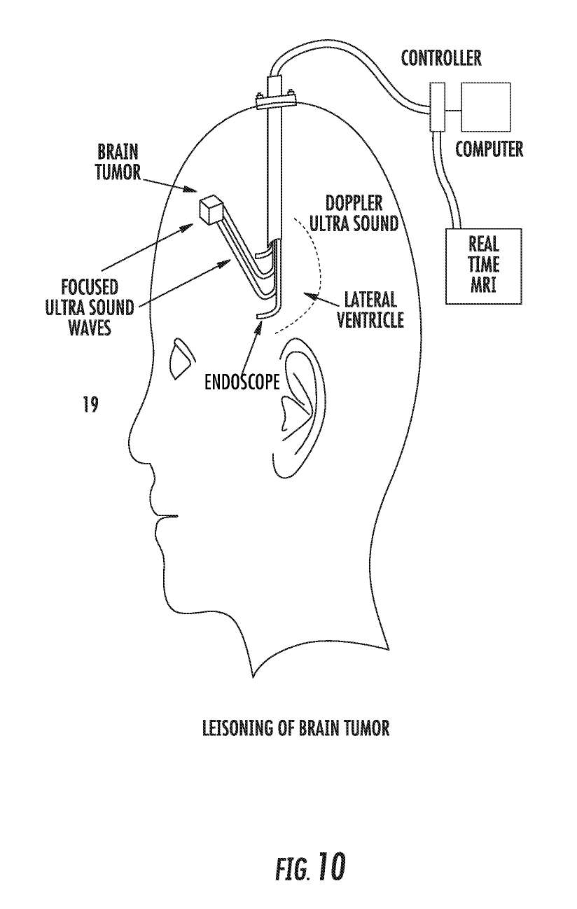

The MIFUS concept will depend on placing the ultrasound transmitters inside of the brain rather than outside as shown in FIG. 9. Earlier ultrasound technology consisted of transmitters that were too large for MIFUS.

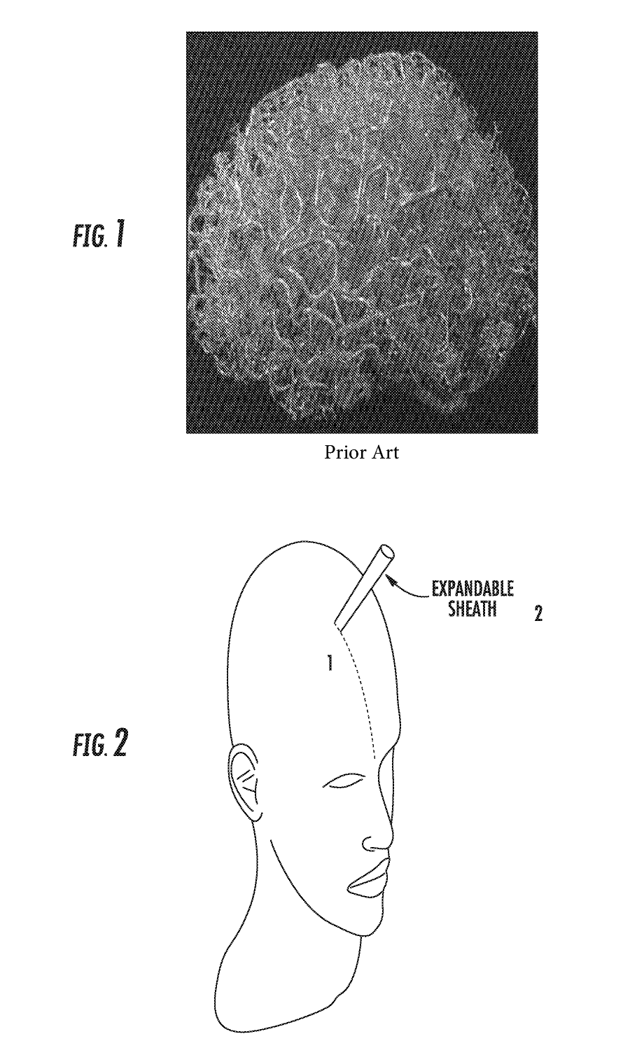

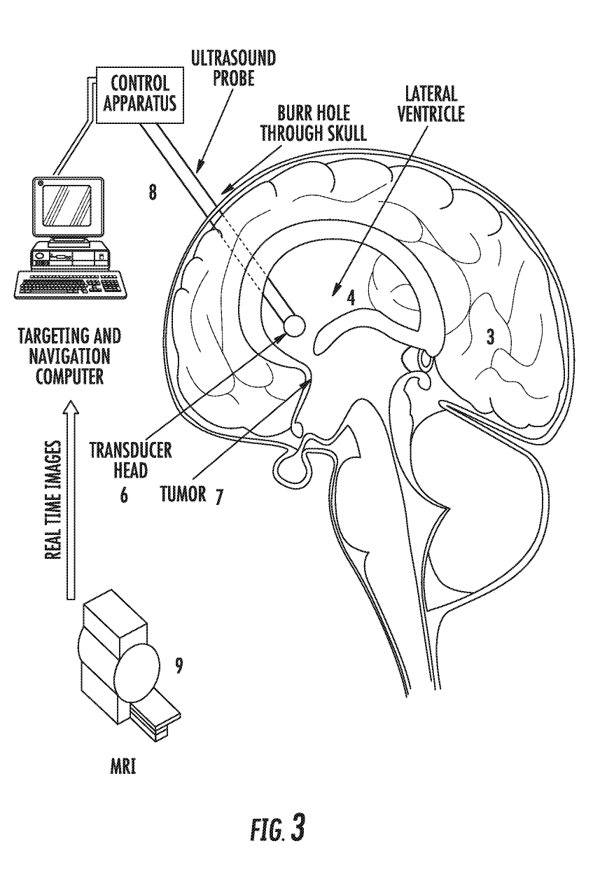

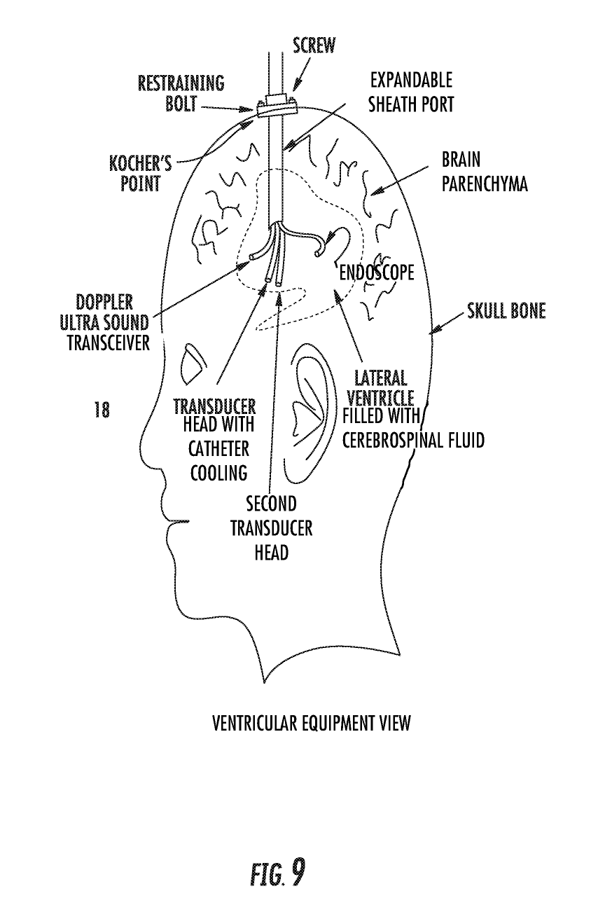

Furthermore, the surgical configuration will have the following features:
Utilize surgical and physical anatomy with micro-technology to minimize bone removal, and brain and blood vessel collateral damage
Insert a small diameter probe through small hole, created at Kocher's point on the skull, into one or both lateral ventricles, see FIG. 2.
Attach stabilization mechanism to support insertion of the expandable sheath, FIG. 8.
Move specially designed micro ultrasound transducer heads through the probe.
The key aspect of the MIFUS system is to use recently available, specially designed micro ultrasound transducers heads having the following specifications:
   Very small 1-5 mm or less;
   Movable/steerable in 3D;
   Can be assembled inside lateral ventricle;
   Can be catheter cooled;
   Can provide sufficient energy intensity and focal length to reach and safely destroy targets within the brain as shown in FIG. 10.
Use currently available video, MRI and ultrasound for real time positioning, thermal dose delivery control and visual data feedback of transducer action, see FIG. 10.
Complete brain exposure is therefore available to multiple focused ultrasound transducers with one small (12 mm) hole; and
   Ratio of exposed brain surface area (targetable) to entry point surface area is extremely high;
   No other current technology can achieve this without having to cross a major amount of bone and or destroy vessels and brain.

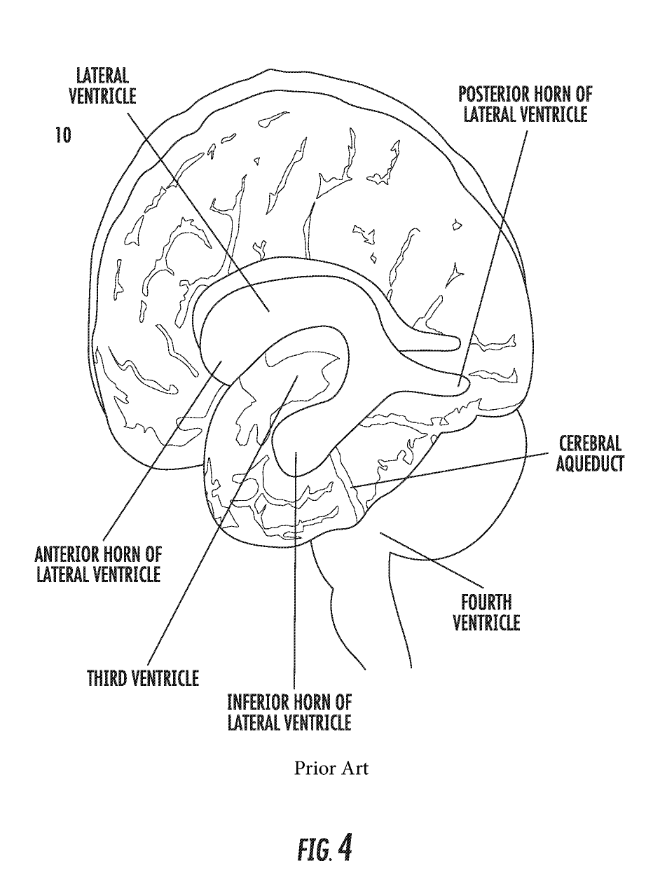

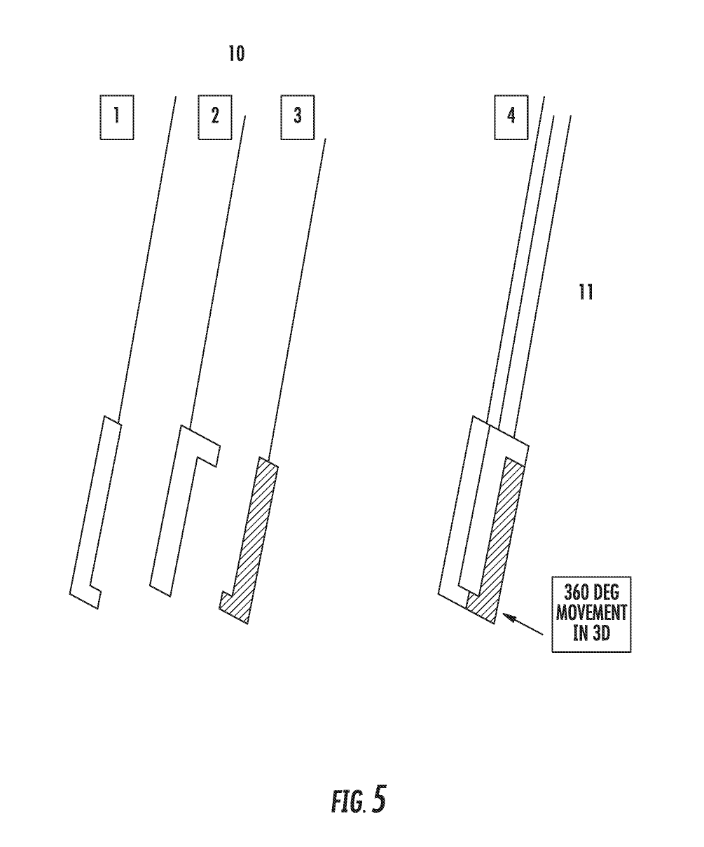

Thus, the lateral ventricle (anterior horn) of the brain is used as a staging point for focused ultrasound. The anatomy of the frontal horn of the lateral ventricle allows device placement with a working ellipsoid volume of 13-15 ml (frontal horn diameter 15 mm×51 mm). Interior use of the ultrasound avoids the bone heating problem as depicted in FIG. 4. Interior placement of the transducers also allows “safe room” to work inside the brain for device assembly as well as allowing targeting of virtually any point in the brain. This approach protects important neural tissue and avoids risk to major blood vessels.

The MIFUS system is organized into the following three functions.
1. Targeting, navigation and dose delivery calculations software
2. Real time visual feedback using MRI and/or Ultrasound
3. Ultrasound delivery components and software

Target Patient Population
Potential patients which may benefit from focused ultrasound treatments in brain include:
   Brain tumors (pediatric and adult, benign and malignant), see FIG. 1;
   Metastasis to brain;
   Brain abscesses;
   Vascular Lesions—Intravascular clot lysis in stroke, AVMs, cavernous malformations, telangiectasias;
   Deep brain stimulation targets (STN and BG targets);
   Epilepsy (medically refractory);
   Blood brain barrier disruption for targeted drug delivery (theoretical);
   Depression.

Targeting, Navigation and Dose Delivery Calculations Software

The MIFUS computer software will accomplish the following functions as shown in FIG. 3:
Planning software for probe navigation, for transducer(s) positioning and stabilization inside skull
   Assembly plan program calculates most efficient plan
   Calculation of transducer number and other needed surgical elements (pre-surgical planning)
   Real time intraoperative ventricular video images guiding plan and operation in real time using stereotactic approaches
Targeting software for optimally guiding energy to a precise area, or areas, of targets, at precise times, with minimal energy delivery to the surrounding tissue used for:
   Navigation software—Distance from transducer to target
   Positioning of heads
   Ablation program—Depends on blood flow to target, surgeons desires etc.
   Determining needed frequency
Ablation monitoring software (all in real time)
   Displays color coded temperature map of target
   Displays ultrasound and Doppler blood flow to target
   Displays color coded map of transducers
   Display fused images: MRI anatomy and color coded thermal ablation

Real Time Visual Feedback

Visual feedback from inside the brain involves the use of MRI and Doppler ultrasound. Characteristics besides those described above with the aid of the computer are as follows, see FIGS. 3 and 10.
MRI allows real time images of target and transducer, positions, sizes and temperatures
Can account for “brain shift”, movement of structures and changes in blood flow intraoperatively
Ultrasound allows real time images of target, Doppler blood flow and elasticity (ultrasound elastography) analysis of target vessels

Focused Ultrasound Delivery Components

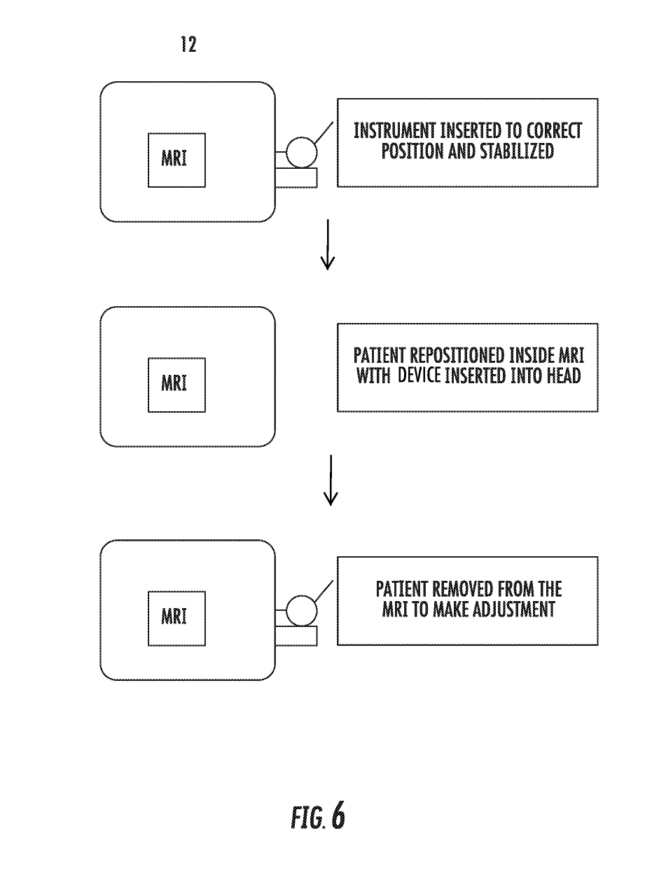

The ultrasound transducers will reside at the distal end of the entry port sheath inside the lateral ventricle anterior horn. The sheath is a 3 mm mesh tube which can be expanded up to 1.2-1.5 cm. An endo scope with optional, fiber optic capability will be inserted to facilitate transducer construction and heads position (1 mm diameter). The sheath is inserted through a small hole in the skull at Kocher's point. The ultrasound transducers have the following characteristics:
Transducer Heads
   Multiple heads with pseudo 3D maneuverability;
   Can be assembled inside ventricle, see FIG. 5;
   Closed loop cooling circuitry;
   Very small but powerful enough to provide up to 50 W/cm2 intensity;
   MR compatible.
Calculated example field parameters of transducer heads in water with intensity up to 300 W/cm2

| | | | Focal |
|---|---|---|---|
| | Transducer | Focal | Length |
| Frequency | Diameter | Length | Max |
| (MHz) | (mm) | Min (mm) | (mm) |
| 2.25 | 6.35 | 8.9 | 11.4 |
| 2.25 | 9.5 | 12.7 | 26.9 |
| 3.5 | 6.35 | 9.8 | 17.8 |
| 3.5 | 9.5 | 15.24 | 41.9 |
| 5 | 6.35 | 10.9 | 25.4 |
| 5 | 9.5 | 15.24 | 59.7 |
| 10 | 6.35 | 16.7 | 53.3 |
| 10 | 9.5 | 15.24 | 120.6 |
| 15 | 6.35 | 12.7 | 80.0 |
| 15 | 9.5 | 15.2 | 180.3 |
| 20 | 6.35 | 12.7 | 106.7 |
| 20 | 3.17 | 6.35 | 25.4 |
| 25 | 6.35 | 12.7 | 133.4 |

Combined MIFUS Operative Device Concept (See FIG. 3)
Utilize brain lateral ventricles as staging point for MIFUS along with micro-technology to avoid skull bone attenuation.
Use video, Magnetic Resonance Imaging (MRI) and ultrasound for real time data feedback.
Complete brain exposure to multiple focused ultrasound transducers with one small (12 mm) hole.
   Exposed brain (targetable) surface area to entry point surface area to is extremely high
   No other current technology can achieve this without having to cross a major amount of bone and or destroy vessels and brain tissue

Kocher's Point
Utilize unique point on skull for entry
Kocher's point is 2-3 cm lateral to the midline of the skull and 1-2 cm anterior to the coronal suture, see FIG. 2:
   Special point which gives a direct line to the lateral ventricle
   Positions device in larger area (anterior horn) of the ventricle but also avoids all major blood vessels and eloquent neural strips
Special because it gives a direct line to the lateral ventricle and also avoids all major blood vessels and eloquent neural strips, such as the pre and post central strips
Burr holes, (12 mm) are typically drilled in the bone at this point for a ventriculostomy (bedside procedure)
A 2-3 mm expandable sheath can be inserted into the ventricle and then expanded to some degree even up to 12 mm

The MIFUS System
Use the lateral ventricle (anterior horn), see FIG. 4, as a staging point for focused ultrasound;
Anatomy of the frontal horn of the lateral ventricle allows device placement with a working ellipsoid volume of 13-15 ml (frontal horn diameter 15 mm×51 mm);
   Avoids bone problem;
   Allows “safe room” to work inside the brain for device assembly;
   Allows targeting of virtually any point in the brain;
   Provides a “fluid buffer” around any potential device;
   Protects important neural tissue;
   Avoids risk to major blood vessels.

Relevant Areas of the Brain
Use specially designed mico-transducer heads
   Very small 1-5 mm
   Movable/steerable in 3D
   Can be assembled inside lateral ventricle
   Can be catheter cooled
   Can provide sufficient energy intensity and focal length to reach and destroy targets

In FIG. 5, the transducer assembly is illustrated. Three parts of a transducer are assembled as shown ultimately providing a 360 degree movement in 3D. These can be magnetic locking mechanisms, mechanical locking mechanisms, electrical locking mechanisms or an operable combination of all of the above.

Entry Port Sheath
3 mm mesh tube which can be expanded up to 1.2-1.5 cm;
Optional, fiber optic scope inserted to facilitate transducer construction and heads position (1 mm diameter).

Computer
Construction Software (used for transducer assembly inside skull)
   Assembly plan program calculates most efficient plan
   Calculation of transducer number and other needed surgical elements (pre-surgical planning)
   Real time intraoperative ventricular video images guiding plan operation in real time
Targeting Software (guides energy to precise area or areas of target at precise times) used for:
   Navigation Software—Distance from transducer to target
   Positioning of Heads
   Ablation program—Depends on blood flow to target, surgeons desires etc.
   Determining needed frequency
Ablation Monitoring Software (all in real time)
   Displays color coded temperature map of target
   Displays ultrasound and Doppler blood flow to target
   Displays color coded map of transducers
   Display fused images: MRI anatomy and color coded thermal ablation

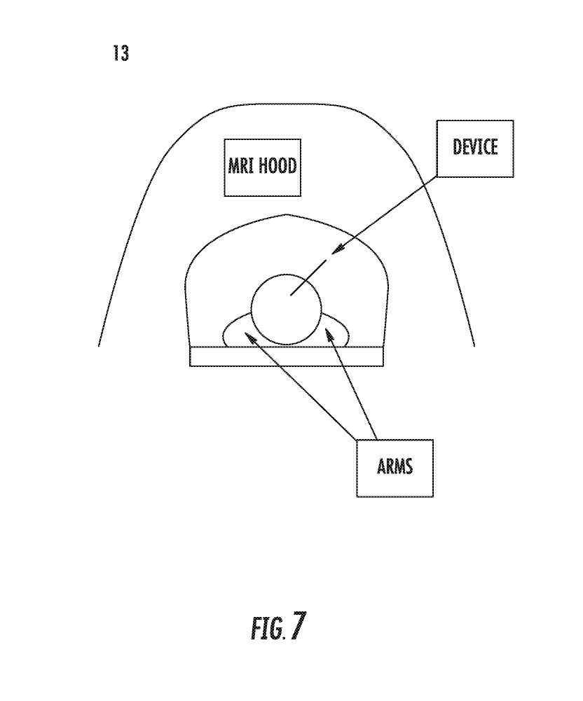

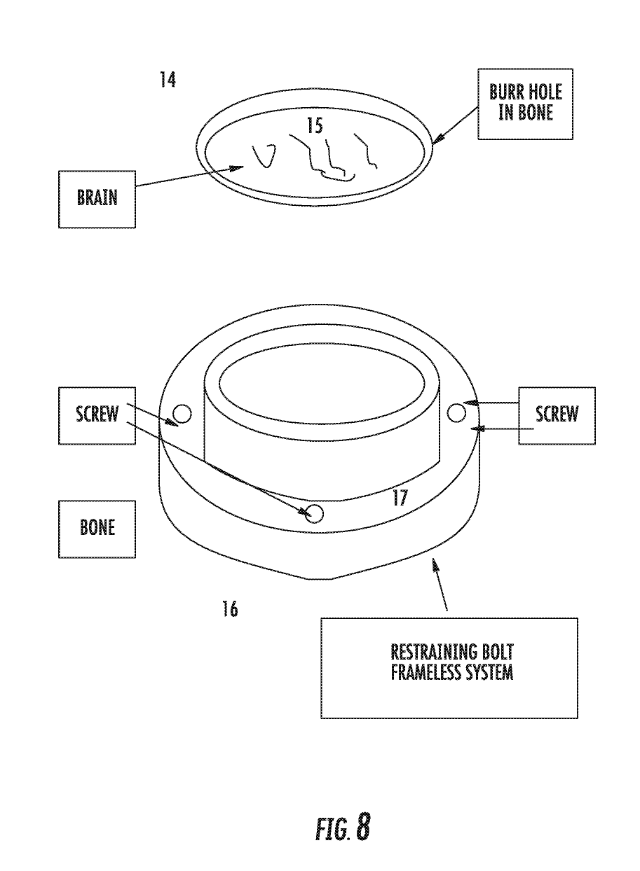

MRI and Ultrasound (See FIGS. 6 and 7)
MRI allows real time images of target and transducer, positions, sizes and temperatures
Ultrasound allows real time images of target and Doppler blood flow analysis of target vessels

Problems Solved/Advantages of MIFUS
Benefit from the same focused ultrasound abilities as external transducers without skull bone attenuation problem.
Allow focused ultrasound targeting of any area of the brain with multiple transducer heads in tandem from multiple angles (steerable heads).
Safety—Do not destroy important neural tissue or cause major bleeding
Versatility—Usable for many different indications, multiple lesions per surgery, multiple thermal ablation options, multiple transducer heads placed one or both ventricles.
Procedure relatively inexpensive compared to the surgery and therefore market competitive (no need for long time hospitalization).
Minimally invasive: creates 1 very tiny 12 mm hole in bone, and is hands free.
Avoid major blood vessels and bleeding risk.
Avoids destruction of vital brain tissue.
Avoids radio-therapy of brain tissue.
Allows targeting of multiple lesions in a single procedure (metastasis).
Allows almost any conceivable three dimensional targeting to a lesion, not just a standard head on approach (Radio Frequency, RF, or laser).
Allows independent ablation planning by surgeon tailored to condition.
Keep temperature rise within a very small area.
Minimal healing time for patient.

Examples of Intended Use
Brain Tumors (including metastasis);
Brain abscesses;
Brain, ventricular and spinal cysts;
Vascular lesions (Arteriovenous malformation, AVMs, cavernous malformations and capillary telangiectasias), clot (stroke), aneurysms;
Part of lesioning for Deep Brain Stimulation, DBS, to treat Parkinson's Disease, PD, essential tremor and epilepsy.
Targeted drug delivery systems

Summary of MIFUS Advantages
The idea uses new a specially designed device which can deliver high intensity focused ultrasonic waves to virtually any part of the brain with little risk to the patient. The tissue destruction is monitored in real time and controlled in a precise manner.
The device utilizes new engineering technology not yet seen in any current surgical/medical device and takes advantage of anatomical and surgical anatomy to avoid current major problems with brain focused ultrasound
The advantages of this method/device over others are its simplicity, versatility and effectiveness
Its simplicity allows it to be low cost. This cost benefit is realized in terms of low device cost, reduced operation training time and lastly markedly reduced patient recovery/hospitalized time
This device avoids pitfalls of recent or earlier methods which attempted to use focused ultrasound in brain and circumvents many of the issues that made these earlier methods unsuccessful.

What is claimed is:

1. A system comprising:
a. an entry port sheath defined as a 3 mm mesh tube which can be expanded up to 1.2-1.5 cm diameter and is used as a port of entry once the burr holes and ventricle tap have been finished, the port being set in a fiberoptic tube having have UV light transmitted intermittently or continuously therein;
b. Micro-transducer ultrasound heads having a size between 1 and 5 mm and
c. a computer configured to calculate an assembly plan program, a component program for calculation of transducer number, and real time ventricular video images guiding plan operation in real time.
